(19) United States
(12) Patent Application Publication
(10) Pub. No.: US 2025/0033052 A1
(43) Pub. Date: Jan. 30, 2025

(54) Device and Method for Splitting Three-Dimensional Agglomerates

(71) Applicant: Robert Bosch GmbH, Stuttgart (DE)

(72) Inventors: Michael Stumber, Korntal-Muenchingen (DE); Bernd Scheufele, Koengen (DE); Tabea Hein, Rothenburg O.D.T (DE); Carina Schuessler, Renningen (DE)

(21) Appl. No.: 18/717,105

(22) PCT Filed: Nov. 22, 2022
(86) PCT No.: PCT/EP2022/082746

§ 371 Date: Jun. 6, 2024

(30) Foreign Application Priority Data
Dec. 14, 2021 (DE) 10 2021 214 276.1

(51) Int. Cl. B01L 3/00; C12M 3/06

(52) U.S. Cl. CPC B01L 3/502761; C12M 23/16; B01L 2200/0647; B01L 2300/0681; B01L 2300/0864; B01L 2400/0487; B01L 2400/06

(57) ABSTRACT

A microfluidic device for mechanically splitting, in particular aided by enzymatic cleaving, of three-dimensional agglomerates to individual structures and/or agglomerate fragments is disclosed. The device has a first fluidic connection and a second fluidic connection and a first microfluidic channel which is located between the first and the second fluidic connection and which has at least one narrow portion in which three-dimensional agglomerates can be mechanically split by friction.

The present invention relates to a microfluidic device, a method for operating the same, as well as a processing unit and a cartridge comprising the microfluidic device according to the preambles of the independent claims.

### PRIOR ART

Interest in the use of three-dimensional agglomerates, e.g. organoids or spheroids for the research, diagnosis, and treatment of diseases, such as tumor diseases, has increased sharply over the past few years, as such three-dimensional agglomerates may well reflect, for example, organ-specific characteristics.

Typically, these are handled manually using aids such as pipettes, reaction vessels, and laboratory equipment. In this case, microfluidics provides advantages compared to conventional laboratory tests, such as lower sample volumes and reagents required, reduced analysis times, and parallel operations.

However, given their complexity, there are numerous challenges to be overcome when implementing required process steps into a microfluidic system. One of these challenges is, for example, the cultivation and propagation of such three-dimensional cell agglomerates in a microfluidic system.

What are referred to as lab-on-a-chip systems (abbreviated as LoC systems) are microfluidic systems that accommodate functionalities of a macroscopic laboratory on a plastic substrate for an automated process. Such systems enable biochemical processes to be processed as completely as possible or to be fully automated.

Lab-on-a-chip systems typically comprise two main components. The first is a test carrier, for example in the form of a cartridge, which comprises structures and mechanisms for manipulation of a received sample, in particular passive components such as channels, reaction chambers, or upstream reagents, or also active components such as valves, pumps, or mixers. The second main component is a control unit for controlling the microfluidic flows in the cartridge.

DE 1020122198660 A1 describes a microfluidic module, a system, and a method for analyzing three-dimensional structures. The microfluidic module comprises a module body having an underside that is configured for positioning on an object stage of a microscope. A channel is formed in the module body comprising a narrow portion, whereby the channel is configured for hydrodynamically positioning the three-dimensional structure at the narrow portion as a liquid flows through the channel.

Coplanar film electrodes are arranged at the ends of the channel to perform an impedance analysis of the three-dimensional structure fixed at the narrow portion.

A measuring device, a measurement method, and a high-throughput test device for electrophysiological measurements on cell aggregates are known from DE 10 2017 130 518 A1.

A fixation portion of a microfluidic channel of the measuring device is constricted, such that a spheroid directed into the narrow portion is deformed from a spherical shape to an elongated shape. This serves to ensure that when the cell aggregate is rinsed with an insulating medium, it flows through the cell aggregate with as short a path as possible. Perfusion with an insulating medium serves to prevent leakage currents between the cells of the cell aggregate during electrophysiological measurement.

### DISCLOSURE OF THE INVENTION

For example, what are referred to as tumor organoids and spheroids are used to research tumor diseases. These enable a very good reproduction of various pathological tissue conditions, so they offer themselves for drug testing to evaluate the efficacy and dosage of drugs. These observations can, e.g., be used to select a personalized drug cancer therapy for the individual patient which takes individual characteristics into account and thus optimizes the efficiency of the therapy.

One way of producing tumor organoids is to remove individual cells or tissue fragments from a cancer patient's primary tumor and then cultivate them. In this case, differentiation and propagation of these cells or tissue fragments takes place, which eventually self-organize into three-dimensional structures. Therefore, the resulting tumor organoids are three-dimensional cell agglomerates that have a similar composition and architecture to that of the patient's primary tumor tissue. They have a diameter of, e.g., 30-500 μm.

Spheroids are three-dimensional cell agglomerates that can be generated by the aggregation and organization of several thousand cells having a diameter of 100-800 μm. Compared to organoids, spheroids are less complex and typically comprise cells of only one type.

In order to be able to work with organoids or spheroids over a longer period of several weeks or months, established methods of 3D cell cultivation are currently used.

The main process steps are the cultivation of organoids or spheroids and their subsequent expansion. During expansion, the organoids or spheroids are enzymatically cleaved and/or mechanically split into organoid or spheroid fragments of a few tens of cells and into individual organoid cells or spheroid cells. The split organoid or spheroid fragments and cells are then reseeded. In this way, the organoids or spheroids propagate.

The term “splitting” in the context of the present invention is thus understood to mean a dissolution of connections between individual structures of a three-dimensional agglomerate, and the resulting dissociation of the three-dimensional agglomerate into agglomerate fragments and/or individual structures.

The term “three-dimensional agglomerates” in the context of the present invention is, e.g., understood to mean cell agglomerates, such as organoids or spheroids.

Thus, in a splitting process, for example, there is a dissolution of connections between cells of a cell agglomerate and the resulting dissociation of the cell agglomerate into multicellular cell agglomerate fragments or individual cells takes place.

When splitting three-dimensional agglomerates, such as organoids and spheroids, several steps are required to add and separate the three-dimensional agglomerates from various liquids, such as culture media, enzyme solutions or rinsing liquids such as wash buffers, with subsequent resuspension in further liquids, as well as, for example, pipetting the three-dimensional agglomerates up and down for mechanical splitting. On a laboratory scale, the vessels are changed and centrifugation steps and pipetting operations to be monitored visually are achieved. These process steps for cultivation and expansion are very labor-intensive and time-consuming and can only be performed by trained and experienced specialist personnel in appropriately equipped laboratories.

The present invention addresses a microfluidic implementation of the processes for splitting three-dimensional agglomerates, such as tumor organoids or tumor spheroids.

According to the invention, a microfluidic device for mechanically splitting, in particular aided by enzymatic cleaving, of three-dimensional agglomerates into individual structures and/or agglomerate fragments having a first fluidic connection and a second fluidic connection and a first microfluidic channel which is located between the first and second fluidic connection, as well as a method for operating the same are provided with the features of the independent claims.

This is based in particular on the fact that the first microfluidic channel of the microfluidic device has at least one narrow portion at which three-dimensional agglomerates can be mechanically split by friction.

Advantageously, the microfluidic device can be used to perform the working steps for splitting three-dimensional cell agglomerates in an automated manner. As a result, much time is saved due to the elimination of manual work steps, shortened analysis times, and parallel processes. Furthermore, these work steps can therefore not only be performed by trained and experienced skilled personnel, which relieves the workload.

The automation of process steps, in turn, enables their standardization, which offers great advantages, especially for clinical and industrial applications, for example in the field of drug testing and personalized medicine.

It is further advantageous that the three-dimensional agglomerates and the agglomerate fragments, as well as the individual structures, remain viable and cultivatable.

As a result of the process steps being performed within the microfluidic device, the risk of undesirable or problematic contamination of the sample is also reduced.

Further advantageous embodiments of the microfluidic device result from the dependent claims.

In one advantageous embodiment, the at least one narrow portion is formed by a, in particular continuous, streamlining of the diameter of the first microfluidic channel. In this embodiment, the first microfluidic channel is, e.g., tubular in shape, resulting in a round cross-section. Alternatively, the at least one narrow portion is formed by a, in particular continuous, streamlining of the width and/or height of the first microfluidic channel. In this embodiment, the first microfluidic channel is, e.g., angular, thus resulting in a predominantly square or rectangular cross-section.

The advantage of a, in particular continuous, streamlining of the first microfluidic channel to form the narrow portion can be easily achieved in this way by means of production technology. A continuous streamlining of the first microfluidic channel offers the advantage that the narrow portion has no edges where the individual structures of the three-dimensional agglomerates can break up or be destroyed, but the connections between the individual structures of the three-dimensional agglomerates can nevertheless be released.

In one advantageous embodiment, the cross-section of the narrow portion in a central region of the narrow portion is streamlined to 1/10 of the cross-section of the first microfluidic channel.

In a further advantageous embodiment, the at least one narrow portion of the first microfluidic channel has a curved shape. Furthermore, the first microfluidic channel can be meander-shaped and/or the at least one narrow portion of the first microfluidic channel is meander-shaped.

For example, the cross section of the microfluidic channels is rectangular or square, whereby the corners can be rounded. Alternatively, the cross section of the microfluidic channels is, e.g., round or oval.

In a further advantageous embodiment, the first microfluidic channel, in particular a first portion of the first microfluidic channel comprising the at least one narrow portion, can be temperature-controlled, in particular heated and/or cooled.

For this purpose, the microfluidic device comprises, e.g., a heating means which heats or cools the first microfluidic channel, and in particular a first portion of the first microfluidic channel comprising the at least one narrow portion.

Advantageously, an optimal temperature for splitting the three-dimensional agglomerates can be provided in this way.

An optimal temperature for splitting depends (among other things and for example) on the cultivation of the three-dimensional agglomerates. If organoids are cultivated in Matrigel® (Corning), then it is, e.g., advantageous to cool the first microfluidic channel, and in particular a first portion of the first microfluidic channel comprising the at least one narrow portion, because Matrigel liquefies at temperatures of approximately 4° C.

In an embodiment in which enzymatic cleaving of the three-dimensional agglomerates is aided by mechanical splitting at the at least one narrow portion of the first microfluidic channel, the enzymatic cleaving can be further improved by adjusting an optimal effective temperature of the enzyme.

For example, in the case of enzymatic cleaving of organoids and/or spheroids, the enzyme-containing solution comprises the enzyme trypsin or TrypLE™ Express Enzyme (Thermofisher), whose optimal effective temperature is 37° Celsius.

Furthermore, in one embodiment, it is advantageous if the microfluidic device comprises a first retaining element which is formed from semi-circular trapping structures and/or from trapping structures in the form of posts, trays, or pots.

Advantageously, the three-dimensional agglomerates are retained by the specified trapping structures when passing through the first microfluidic channel near the at least one narrow portion, so that they are in the correct position for subsequent splitting.

Within the scope of this application, the term “semi-circular trapping structures” are understood to mean continuous or non-continuous semi-circular obstacles, which are arranged in the first microfluidic channel side-by-side and/or in a row such that the three-dimensional agglomerates are held in the semicircles and cannot pass through the interspaces between the semicircles or, in the case of non-continuous semicircles, the interspaces between the semicircles themselves in the unsplit state.

In the context of this application, trapping structures in the form of posts are to be understood to mean several pillars or posts arranged next to each other, whereby the three-dimensional agglomerates do not fit through the interspace between the individual posts due to their size, so that only split individual structures or agglomerate fragments can pass through the interspace.

In the context of this application, trapping structures in the form of trays or pots are understood to mean recesses of the first microfluidic channel that have the form of elongated trays or round pots into which the three-dimensional agglomerates sink so that they do not pass further through the microfluidic channel.

In an alternative, preferred embodiment, the first retaining element is designed as a microfilter, and/or as a microsieve, and/or as a microstructured lattice with pores. The pores have a diameter of, e.g., 20-80 μm, which is smaller than the diameter of the three-dimensional agglomerates.

Therefore, such pore diameters are particularly advantageous for retaining the three-dimensional agglomerates on the first retaining element because the three-dimensional agglomerates cannot pass through the first retaining element in the unsplit state.

In this way, it is ensured that only split agglomerate fragments or individual structures as well as fluids can pass through the microfluidic device further. It is also advantageous for the three-dimensional agglomerates to collect on or upstream of the first retaining element, in which case they are in an optimal starting position for subsequent splitting at the narrow portion of the first microfluidic channel.

Alternatively, the pores of the first retaining element can have a different pore diameter adapted to the particular application.

A further advantageous embodiment provides that the microfluidic device comprises a second retaining element designed as a microfilter, and/or as a microsieve, and/or as a microstructured lattice having pores. The pores in particular have a diameter of 2-8 μm, and preferably 2-6 μm.

The second retaining element has a pore diameter smaller than the diameter of the individual structures of the three-dimensional agglomerates. The individual structures and agglomerate fragments therefore cannot pass through and accumulate on the second retaining element. Advantageously, fluid exchange is enabled by the second retaining element, but individual structures and agglomerate fragments are retained so that they cannot unintentionally leave the microfluidic device and are therefore not lost.

In a further advantageous embodiment, it is provided that optical and/or microscopic analysis of the split individual structures and/or agglomerate fragments can be performed on the retaining element.

The advantage thereby is that the result of the method for splitting the three-dimensional agglomerates can be analyzed directly on the retaining element. For example, the size of the agglomerate fragments can be analyzed, the amount of the individual structures can be estimated, and/or the viability of the individual structures and agglomerate fragments can be analyzed. For this purpose, the individual structures and/or agglomerate fragments are, e.g., colored on the retaining element by feeding a coloring solution. Alternatively, the three-dimensional agglomerates are colored before being fed to the microfluidic device.

In a further advantageous embodiment, the microfluidic device comprises a second microfluidic channel, which opens up from a top side of the second retaining element in a third microfluidic connection.

Advantageously, the individual structures can thus be transported separately via the second microfluidic channel and can be discharged via the third microfluidic connection. As a result, the microfluidic device is more flexible and complex, enabling more flexible handling and the performance of more complex microfluidic operations.

Furthermore, in one embodiment, it is advantageous for the device to comprise at least one reservoir for a fluid. Advantageously, fluids such as media, rinsing liquids, or enzyme-containing solutions can be stored in the reservoir. This ensures a fast and easy feed of these fluids.

In a further advantageous embodiment, the microfluidic device comprises at least one valve and/or at least one pump, which are in particular electrically controllable, so that the microfluidic device is electrically operable.

Advantageously, at least one fluidic connection and/or at least one microfluidic channel and/or at least one reservoir for fluids can be individually closed and opened by means of one or more valves, so that the flow through the microfluidic device can be individually established. In this way, various ways for the feed and discharge of media as well as the flow through the microfluidic channels can be achieved.

In an alternative embodiment, the at least one valve is arranged outside of the microfluidic device.

In one advantageous embodiment, the feed and discharge of the fluids and/or conveyance of the fluids is performed by at least one pump. The at least one pump is, e.g., a microfluidic pump, so that the complex and complex fluidic connection of the microfluidic device to an external pump is eliminated. In this way, the fluidic feed, discharge, and conveyance processes can be easily and straightforwardly achieved, and the microfluidic device can be designed to be space-saving, portable, and mobile.

In an alternative embodiment, the at least one pump is arranged outside of the microfluidic device. The at least one pump is, e.g., a syringe pump or a peristaltic pump.

The object of the invention is furthermore a method for the mechanically splitting, in particular aided by enzymatic cleaving, of three-dimensional agglomerates to individual structures and agglomerate fragments by means of the microfluidic device according to the invention, said method comprising the following steps:
a) feeding a first medium having three-dimensional agglomerates via a first fluidic connection
b) conveying the first medium comprising three-dimensional agglomerates via a first microfluidic channel
c) moving the first medium comprising three-dimensional agglomerates back and forth in a pulsatile manner through the at least one narrow portion of the first microfluidic channel, whereby the three-dimensional agglomerates are mechanically split at the narrow portion by friction.

The expression “back and forth movement of the first medium in a pulsatile manner” means that the flow direction of the first medium is temporarily changed by the microfluidic device by successive intermittent forward and backward conveying of the first medium.

Advantageously, the three-dimensional agglomerates come into physical contact with the channel inner walls of the narrow portion of the first microfluidic channel due to the back and forth movement of the first medium in a pulsatile manner. Additional friction is created between the three-dimensional agglomerates and the back and forth movement of the first medium in a pulsatile manner. This takes up a much higher speed than the three-dimensional agglomerates, in particular in the narrow portions, which also leads to the washing away or detachment of individual structures or agglomerate fragments on the surface of the three-dimensional agglomerates. In addition, the three-dimensional agglomerates themselves also meet in the narrow portion, thereby creating further friction. These frictional processes cause the connections between the individual structures, which are responsible for the cohesion of the three-dimensional agglomerate, to become increasingly loose. More and more individual structures and agglomerate fragments split off from the three-dimensional agglomerates. In this way, the three-dimensional agglomerates are mechanically dissociated. The individual structures themselves are not damaged and remain viable and cultivatable. The individual structures and agglomerate fragments can then be reseeded and cultivated and grown into three-dimensional agglomerates.

Advantageously, the microfluidic device can be used to perform the work steps for splitting three-dimensional cell agglomerates automatically. Much time is saved due to the elimination of manual work steps, shortened analysis times, and parallel processes. Furthermore, these work steps can not only be performed by trained and experienced skilled personnel, which relieves the workload.

The automation of process steps, in turn, enables their standardization, which offers great advantages, especially for clinical and industrial applications, for example in the field of drug testing and personalized medicine.

As a result of the process steps being performed within the microfluidic device, the risk of undesirable or problematic contamination of the sample is also reduced.

Further advantageous embodiments of the method according to the invention result from the dependent claims.

The first medium comprising the three-dimensional agglomerates is moved back and forth through the at least one narrow portion of the first microfluidic channel at a flow rate of, e.g., 10-80 μL/s. The flow rate is, e.g., constant or varying, in particular pulsating.

In an advantageous embodiment of the microfluidic method, the three-dimensional agglomerates in step b) are conveyed through a first portion of the first microfluidic channel up to a first retaining element, which retains them in unsplit form.

Advantageously, the three-dimensional agglomerates cannot pass through the retaining element in the unsplit state. In this way, it is ensured that only split agglomerate fragments or individual structures as well as fluids can pass through the microfluidic device further. In addition, it is advantageous for the three-dimensional agglomerates to collect on or upstream of the first retaining element, whereby they are in an optimal starting position for subsequent splitting at the narrow portion of the first microfluidic channel.

In a further advantageous embodiment, the following step is performed after step b):
b′) feeding and conveying a first rinsing liquid via the first microfluidic channel for washing the three-dimensional cell agglomerates.

The first rinsing liquid removes any residues of the first medium and cleans the three-dimensional agglomerates as well as the microfluidic device.

In one embodiment, in which the mechanical splitting of the three-dimensional agglomerates performed to aid enzymatic cleaving, a washing step by means of a first rinsing liquid is advantageous as this removes proteins of the first medium adherent to the three-dimensional agglomerates, which otherwise inhibit, for example, the enzyme reaction.

A further advantageous embodiment of the microfluidic method provides that after step b) or after step b′), the following step is performed:
b″) feeding and conveying an enzyme-containing solution via the first microfluidic channel, and whereby, in step c), the enzyme-containing solution comprising the three-dimensional agglomerates is moved back and forth in a pulsatile manner through the at least one narrow portion of the first microfluidic channel such that the enzymatic cleaving of the three-dimensional agglomerates is mechanically aided by friction of the three-dimensional agglomerates at the narrow portion.

For this purpose, the enzyme-containing solution having a splitting effect is, e.g., first added to the three-dimensional agglomerates until the enzyme-containing solution is at least in the area between the first fluidic connection and the first retaining element. The three-dimensional agglomerates are then incubated with the enzyme-containing solution for, e.g., a period of ten minutes. During incubation or thereafter, the enzyme-containing solution comprising the three-dimensional agglomerates is moved back and forth in a pulsatile manner through the at least one narrow portion of the first microfluidic channel to mechanically aid the splitting process. The enzymatic and mechanical action removes individual structures and agglomerate fragments from the three-dimensional agglomerates, so that the splitting process operation is mechanically aided, improved and in particular accelerated.

In a further advantageous embodiment, the first microfluidic channel, and in particular the first portion of the first microfluidic channel comprising the at least one narrow portion, is temperature-controlled. For this purpose, a heating element is, e.g., integrated into the microfluidic device. The heating or cooling is, e.g., performed before and/or during step c) of the microfluidic process so that the first microfluidic channel, and in particular the first portion of the first microfluidic channel comprising the at least one narrow portion, is at the desired temperature during the splitting process.

If enzymatic cleaving within the microfluidic device is aided by mechanical splitting at the at least one narrow portion of the first microfluidic channel, the enzymatic cleaving is further improved by setting an optimal effective temperature of the enzyme.

In an alternative or additional embodiment, ultrasonic waves and/or vibrations are generated that act on the three-dimensional agglomerates in the first microfluidic channel, and in particular in the first portion of the first microfluidic channel having the at least one narrow portion, and that aid and improve the splitting process.

In a further advantageous embodiment, the following step is performed after step c):
d) conveying the split individual structures and/or agglomerate fragments through the first retaining element via a second portion of the first microfluidic channel to a second retaining element, whose pores have a diameter smaller than the diameter of the split individual structures and agglomerate fragments, such that they are retained.

The individual structures and agglomerate fragments cannot pass through the second retaining element and accumulate on or in front of the second retaining element. Advantageously, the individual structures and agglomerate fragments are retained and cannot leave the microfluidic device unintentionally and are therefore not lost. Fluids, on the other hand, can pass through the second retaining element so that a fluid exchange can occur.

In one embodiment, it is advantageous if the spliced individual structures and agglomerate fragments are conveyed through the first retaining element via the second portion of the first microfluidic channel to the second retaining element in a second rinsing liquid, which is fed to the first microfluidic channel and conveyed via this. Advantageously, for example, the enzyme-containing solution is removed from the three-dimensional agglomerates and the splitting process is stopped in this way.

Furthermore, it is advantageous in one embodiment if the following step is performed after step d):
e) conveying the individual structures and/or agglomerate fragments back via the first microfluidic channel and discharging the individual structures and/or agglomerate fragments via the first fluidic connection, which serves in this case as an outlet.

Advantageously, the microfluidic device in this case has a simple construction and no additional components, e.g. reservoirs or further optional channels and structures, for transporting the individual structures and agglomerate fragments are required.

In an alternative advantageous embodiment, the following step is performed after step d):
e′) feeding a second medium via a second microfluidic connection, which serves in this case as an inlet, and guiding the second medium via a third portion of the first microfluidic channel to an underside of the second retaining element and through the latter such that the retained individual structures and/or agglomerate fragments on the top side of the second retaining element are transferred with the second medium into a second microfluidic channel, which opens up in a third fluidic connection, and discharging the individual structures and/or agglomerate fragments via the third fluidic connection, which serves in this case as an outlet.

Advantageously, the individual structures and agglomerate fragments do not have to pass the first and second portions of the first microfluidic channel again, but are performed via a closer third fluidic connection. As a result, the microfluidic method for mechanically splitting three-dimensional agglomerates can be repeated with new three-dimensional agglomerates fed via the first fluidic connection,

whereas the individual structures and/or agglomerate fragments are discharged at regular intervals via the third fluidic connection. This prevents overloading or clogging of the second retaining element by too large a number of accumulating individual structures and/or agglomerate fragments.

In one particularly advantageous embodiment, the three-dimensional agglomerates are cell agglomerates, in particular organoids or spheroids, and the split individual structures are cells, in particular organoid cells or spheroid cells and the agglomerate fragments are cell agglomerate fragments, in particular organoid fragments or spheroid fragments.

Advantageous when using cell agglomerates, for example organoids or spheroids, is that their viability is well maintained, thus enabling further cultivation and expansion steps.

A further object of the invention is a control unit for controlling the microfluidic method according to the invention, in particular by electrical actuation of the at least one valve and/or the at least one pump.

A further object of the invention is a cartridge, in particular a microfluidic cartridge, as described in, e.g., DE102016222072A1 or DE102016222075A1, comprising the microfluidic device according to the invention.

### BRIEF DESCRIPTION OF THE DRAWINGS

Embodiments of the present invention are illustrated in the drawings and further explained in the subsequent description thereof. Shown are:

FIG. 1: the schematic illustration of a view of a microfluidic device according to the invention in a first embodiment comprising a first microfluidic channel having narrow portions,

FIG. 2: the schematic representation of a three-dimensional technical drawing of a microfluidic device according to the invention in a second embodiment having a first microfluidic channel comprising narrow portions and a second microfluidic channel,

FIG. 3: the schematic illustration of a cartridge according to the invention comprising the microfluidic device according to FIG. 1, and

FIG. 4: the schematic representation of an exemplary embodiment of the method according to the invention.

### EMBODIMENTS OF THE INVENTION

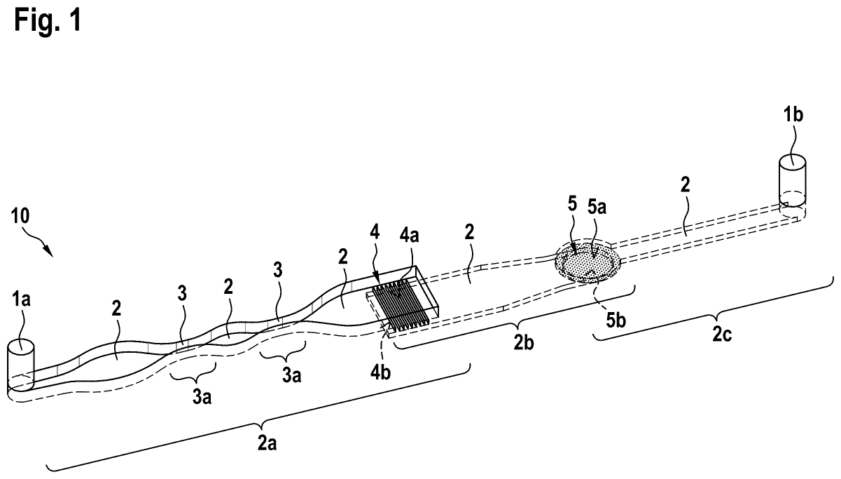

FIG. 1 shows a first embodiment of the microfluidic device 10 according to the invention for mechanically splitting three-dimensional agglomerates.

The microfluidic device 10 comprises a first fluidic connection 1a and a second fluidic connection 1b. A first microfluidic channel 2 is arranged between the first fluidic connection 1a and the second fluidic connection 1b. The first microfluidic channel 2 comprises a first section 2a, a second section 2b, and a third section 2c.

The first microfluidic channel 2 in the first section 2a comprises two narrow portions 3, at which three-dimensional agglomerates can be mechanically split by friction.

Alternatively (and not shown in FIG. 1), the first microfluidic channel 2 can also comprise only one narrow portion 3 or a plurality of narrow portions 3.

The narrow portions 3 are formed by streamlining the width of the first microfluidic channel 2. The streamlining of the width of the first microfluidic channel 2 is continuous from both directions proceeds towards the central area 3a of the narrow portion 3. In the central area 3a of the narrow portion 3, the first microfluidic channel 2 has a streamlined design with a constant width.

Alternatively (and not shown in FIG. 1), the streamlining area of the narrow portion 3 directly transitions to a widening area such that narrow portion 3 does not include a constant width area. The first microfluidic channel 2 can be temperature-controlled in the first section 2a, which comprises the narrow portions 3. For example, the microfluidic device 10 comprises a heating element for this purpose (not shown in FIG. 1).

The microfluidic device 10 comprises a first retaining element 4. For example, the first retaining element 4 is designed as a microfilter and/or as a microsieve and/or as a microstructured lattice with pores. For example, the pores of the first retaining element 4 have a diameter of 20-80 μm.

Furthermore, the microfluidic device 10 comprises a second retaining element 5 designed as a microfilter and/or as a microsieve and/or as a microstructured lattice having pores. For example, the pores of the second retaining element 5 have a diameter of 2-8 μm, and preferably of 2-6 μm.

The first portion 2a of the first microfluidic channel 2 extends from the first fluidic connection 1a to a top side 4a of the first retaining element 4. The second portion 2b of the first microfluidic channel 2 extends from an underside 4b of the first retaining element 4 up to an underside 5b of the second retaining element 5. The third portion 2c of the first microfluidic channel 2 extends from a top side 5a of the second retaining element 5 up to the second fluidic connection 1b.

The microfluidic device 10 also comprises at least one valve (not shown in FIG. 1) and/or pump (not shown in FIG. 1), which are electrically controllable such that the microfluidic device 10 is electrically operable. The microfluidic device 10 further comprises, e.g., at least one reservoir for a fluid (not shown in FIG. 1).

The microfluidic device 10 is designed to perform a method 500 for mechanically splitting three-dimensional agglomerates into individual structures and agglomerate fragments.

The following is an exemplary description of mechanically splinting organoids in organoid cells and/or organoid fragments to aid enzymatic cleaving.

In a first step a), a first medium (e.g., a solution comprising organoids) is, e.g., fed to the microfluidic device 10 via the first fluidic connection 1a which is stored upstream, for example in a reservoir (not shown in FIG. 1). In a second step b), the first medium comprising the organoids is conveyed via the first microfluidic channel 2. The organoids pass through the narrow portions 3 of the first microfluidic channel 2 and are finally retained by the first retaining element 4, as the diameter of the organoids is greater than the diameter of the pores of the first retaining element 4. The first medium, on the other hand, passes through the first retaining element 4 and the second portion 2b of the first microfluidic channel 2, the second retaining element 5 and the third portion 2c of the first microfluidic channel 2 and is finally discharged via the second fluidic connection 1b. A valve is then, e.g., switched to a different reservoir (not shown in FIG. 1) with a first rinsing fluid.

In a further step b′), the first rinsing liquid is fed to the microfluidic device 10 via the first fluidic connection 1a and conveyed via the first microfluidic channel 2. The first rinsing liquid washes the organoids, thus removing any residues from the first medium.

The first rinsing liquid is, e.g., a phosphate buffered saline (PBS). The first rinsing liquid is finally discharged again via the second microfluidic connection 1b. A valve is then, e.g., switched to a different reservoir (not shown in FIG. 1) with an enzyme-containing solution.

In step b″), the enzyme-containing solution is fed to the microfluidic device 10 via the first fluidic connection 1a and conveyed via the first microfluidic channel 2. For example, the enzyme-containing solution includes the enzyme trypsin or TrypLE™ Express Enzyme (Thermofisher), which results in enzymatic cleaving of the organoids. If the enzyme-containing solution is located in the entire first section 2a of the first microfluidic channel 2, then, in step c), the enzymatic cleaving of the organoids is mechanically aided by a back and forth movement of the enzyme-containing solution comprising the organoids in a pulsatile manner through the narrow portions 3 of the first microfluidic channel 2. Due the action of the enzyme and friction of the organoids at the narrow portions 3 of the first microfluidic channel 2, organoid cells and/or organoid fragments are split off from the organoids until the organoids are in particular completely split into organoid cells and/or organoid fragments from a few tens of organoid cells. The organoid cells and/or organoid fragments then have a diameter smaller than the pore diameter of the first retaining element 4.

Prior to and/or during the splitting process, the first portion 2a of the first microfluidic channel 2 comprising the narrow portions 3 is, e.g., heated in order to in particular adjust an ideal temperature for the enzyme action.

A valve is then, e.g., switched to a different reservoir (not shown in FIG. 1) with a second rinsing fluid.

In a next step d), the second rinsing liquid is fed to the microfluidic device 10 via the first fluidic connection 1a and conveyed via the first microfluidic channel 2. The second rinsing liquid removes the enzyme-containing solution and thus stops the splitting process. The organoid cells and organoid fragments are transported through the first retaining element 4 and via the second portion 2b of the first microfluidic channel 2 with the second rinsing liquid until they are finally retained on a lower side 5b of the second retaining element 5. The pores of the second retaining element 5 have a smaller diameter than the organoid cells and organoid fragments. Only the second rinsing liquid passes through the second retaining element 5 and the third portion 2c of the first microfluidic channel 2 and is finally discharged via the second fluidic connection 1b.

To discharge the organoid cells and organoid fragments, a second medium is supplied to the microfluidic device 10 via the second fluidic connection 1b and conveyed via the first microfluidic channel 2 up to the first fluidic connection 1a, which serves in this case as an outlet. In this case, the second medium first passes through the third section 2c of the first microfluidic channel 2 and the second retaining element 5. The organoid cells and organoid fragments which have accumulated on the underside 5b of the second retaining element 5 are carried along with the second medium via the first microfluidic channel 2 in a step e), such that the organoid cells and organoid fragments are also discharged via the first fluidic connection 1a.

The organoid cells and organoid fragments can be forwarded, divided, and/or optionally reseeded for repeated organoid growth.

Similarly, for example, a method for splitting spheroids in spheroid cells and/or spheroid fragments to aid enzymatic cleaving is performed.

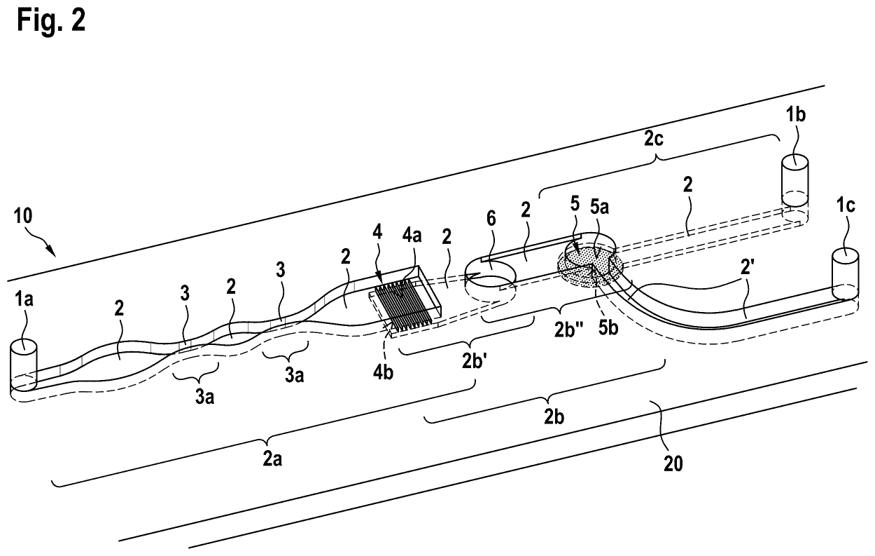

In FIG. 2, the microfluidic device 10 according the invention is illustrated in a second exemplary embodiment. The microfluidic device 10 is constructed in a manner similar to the microfluidic device 10 in FIG. 1, with the differences described hereinafter.

The second portion 2b of the first microfluidic channel 2 is subdivided into two regions—a first region 2b′ of the second portion 2b and a second region 2b″ of the second portion 2b. The first region 2b′ is at a lower level and the second region 2b″ is at a higher level. A structure 6 is arranged between the first region 2b′ and the second region 2b″ of the second section 2b of the first microfluidic channel 2 which forms a vertically oriented passage between the lower first region 2b′ of the second section 2b to the higher level second region 2b″ of the second section 2b.

The microfluidic device 10 further comprises a second microfluidic channel 2″ which opens up from a top side 5a of the second retaining element 5 in a third microfluidic connection 1c.

The second retaining element 5 is arranged in the vertical direction with respect to the higher level and the lower level on an intermediate plane

In FIG. 2, it is indicated by way of example for all embodiments of the microfluidic devices 10 according to the invention that the microfluidic device 10 is, e.g., accommodated on a plastic substrate 20 or chip (as described in the parallel application).

The differences to the method described in FIG. 1 for mechanically splitting organoids in organoid cells and/or organoid fragments to aid enzymatic cleaving are described below using the microfluidic device 10 shown in FIG. 2.

In step d), the split organoid cells and organoid fragments pass through the first retaining element 4 with the second rinsing liquid and, after passing through, enter a first region 2b′ of the second portion 2b of the first microfluidic channel 2, which is located at a lower level. The vertically oriented structure 6 guides the organoid cells and organoid fragments upwards so that they reach a second region 2b″ of the second section 2b, which is at a higher level. From there, the organoid cells and organoid fragments are rinsed directly on the top side 5a of the second retaining element 5, which in turn lies slightly lower on the intermediate level with respect to the higher level and slightly higher with respect to the lower level.

The second retaining element 5 prevents the organoid cells and organoid fragments from passing through, so that they accumulate on its top side 5a. Only the second rinsing liquid passes through the second retaining element 5 and the third portion 2c of the first microfluidic channel 2 and is finally discharged via the second fluidic connection 1b. In this case, the second microfluidic channel 2 is, e.g., closed by a valve (not shown).

To discharge the organoid cells and organoid fragments, the second portion 2b of the first microfluidic channel 2 adjacent to the second retaining element 5 is, e.g., closed by a valve (not shown), and the second microfluidic channel 2 is, e.g., opened by opening a valve (not shown). In a next step e), a second medium is fed via the second fluidic connection 1b, which serves in this case as an inlet. The second medium is conveyed via the third portion 2c of the first microfluidic channel 2, passes the second retaining element 5, whereby it is conveyed from an underside 5b of the second retaining element 5 through the latter to a top side 5a of the second retaining element 5. The organoid cells and organoid fragments which have accumulated on the top side 5a of the second retaining element 5 are carried along with the second medium, fed into the second microfluidic channel 2 and discharged via the third fluidic connection 1c, which in this case serves as an outlet.

The organoid cells and organoid fragments can be forwarded, divided, and/or optionally reseeded for repeated organoid growth.

In the simplest embodiment of the method according to the invention, the microfluidic device 10 that can be used for this purpose (not shown in the drawings) comprises only the first 1a, and the second fluidic connection 1b and a first microfluidic channel 2 arranged between them. In this embodiment, the first microfluidic channel 2 comprises only the first section 2a of the first microfluidic channel 2 comprising at least one narrow portion 3.

In a first step a), a first medium, e.g. a solution comprising three-dimensional agglomerates, is fed to the microfluidic device 10 via the first fluidic connection 1a, for example, which is stored upstream, for example in a reservoir. In a second step b), the first medium comprising the three-dimensional agglomerates is conveyed via the first microfluidic channel 2 until the first medium comprising the three-dimensional agglomerates is located in the first microfluidic channel 2 from the first fluidic connection 1a to the second fluidic connection 1b.

Subsequently, in a further step c), the three-dimensional agglomerates are mechanically split by a back and forth movement of the first medium comprising the three-dimensional agglomerates in a pulsatile manner through the narrow portions 3 of the first microfluidic channel 2. Due to the friction of the three-dimensional agglomerates at the narrow portions 3 of the first microfluidic channel 2, individual structures and/or agglomerate fragments are split off from the three-dimensional agglomerates until the three-dimensional agglomerates are in particular completely split into individual structures and/or agglomerate fragments consisting of a few tens of individual structures.

To discharge the individual structures and agglomerate fragments, the first medium without three-dimensional agglomerates is def to the microfluidic device 10 in a step e) via the second fluidic connection 1b, and this is conveyed via the first microfluidic channel 2 up to the first fluidic connection 1a, which serves in this case as an outlet. The individual structures and agglomerate fragments are carried along with the first medium via the first microfluidic channel 2, such that the individual structures and agglomerate fragments are also discharged via the first fluidic connection 1a.

The individual structures and agglomerate fragments can be forwarded, divided, and/or optionally reseeded for repeated growth of the three-dimensional agglomerates.

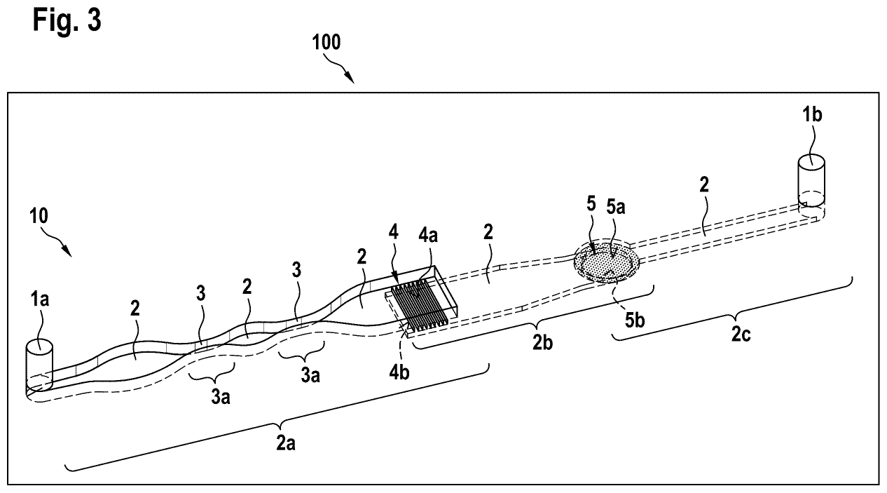

FIG. 3 shows a cartridge 100 according to the invention, which comprises, as an example, all embodiments of the microfluidic device 10 according to the invention, a microfluidic device 10 in the first embodiment according to FIG. 1.

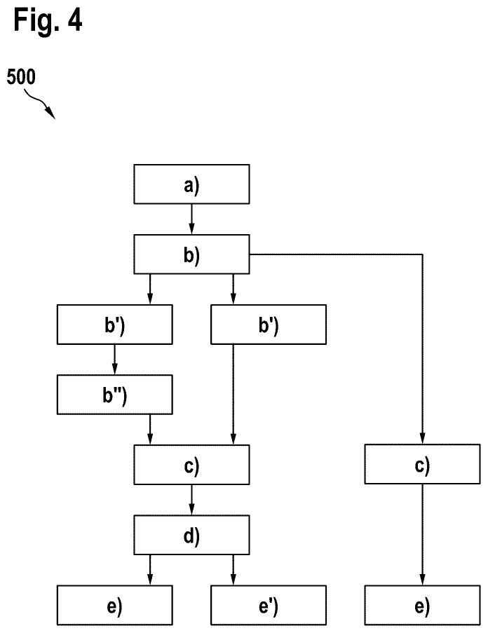

FIG. 4 shows a flow chart with exemplary embodiments of the method 500 according to the invention for mechanically splitting, in particular aided by enzymatic cleaving, of three-dimensional agglomerates to individual structures and/or agglomerate fragments, for example in connection with the exemplary embodiments and method steps described for FIGS. 1 and 2.

1. A microfluidic device for mechanically splitting, aided by enzymatic cleaving, of three-dimensional agglomerates to individual structures and/or agglomerate fragments, the device comprising:
a first fluidic connection;
a second fluidic connection; and
a first microfluidic channel which is located between the first fluidic connection and the second fluidic connection,
wherein the first microfluidic channel has at least one narrow portion which is configured to mechanically split three-dimensional agglomerates by friction.

2. The microfluidic device according to claim 1, wherein the at least one narrow portion is formed by a continuous, streamlining of the diameter or by a continuous, streamlining of the width and/or height of the first microfluidic channel.

3. The microfluidic device according to claim 1, wherein the at least one narrow portion comprises a streamlined central region having a constant diameter or a constant width and/or height.

4. The microfluidic device according to claim 1, wherein a first portion of the first microfluidic channel comprising the at least one narrow portion is configured to be heated or cooled.

5. The microfluidic device according to claim 1, wherein:
the microfluidic device comprises a first retaining element, which is formed from semi-circular trapping structures and/or from trapping structures in the form of posts, trays, or pots, or
the first retaining element is designed as a microfilter, and/or as a microsieve, and/or as a microstructured lattice with pores, the pores having a diameter of 20-80 μm.

6. The microfluidic device according to claim 1, further comprising a second retaining element designed as a microfilter, and/or as a microsieve, and/or as a microstructured lattice with pores, the pores having a diameter of 2-8 μm.

7. The microfluidic device according to claim 6, further comprising a second microfluidic channel which opens up from a top side of the second retaining element into a third microfluidic connection.

8. The microfluidic device according to claim 1, further comprising:
at least one valve and/or pump which is/are electrically controllable such that the microfluidic device is electrically operable; and/or
at least one reservoir for a fluid.

9. A microfluidic method for mechanically splitting, aided by enzymatic cleaving, of three-dimensional agglomerates to individual structures and/or agglomerate fragments by way of a microfluidic device according to claim 1, the method comprising:
a) feeding a first medium comprising three-dimensional agglomerates via the first fluidic connection;
b) conveying the first medium comprising three-dimensional agglomerates via the first microfluidic channel; and
c) moving the first medium comprising three-dimensional agglomerates back and forth in a pulsatile manner through the at least one narrow portion of the first microfluidic channel so that the three-dimensional agglomerates are mechanically split by friction at the narrow portion.

10. The microfluidic method according to claim 9, wherein the three-dimensional agglomerates in step b) are conveyed through a first portion of the first microfluidic channel to a first retaining element, which retains them in an unsplit form.

11. The microfluidic method according to claim 9, wherein, after step b), the following step is performed:
b′) feeding and conveying a first rinsing liquid via the first microfluidic channel in order to wash the three-dimensional agglomerates.

12. The microfluidic method according to claim 9, wherein:
after step b) or after step b′), the following step is performed: b″) feeding and conveying an enzyme-containing solution via the first microfluidic channel, and
in step c), the enzyme-containing solution comprising the three-dimensional agglomerates is moved back and forth in a pulsatile manner through the at least one narrow portion of the first microfluidic channel such that the enzymatic cleaving of the three-dimensional agglomerates is mechanically aided by friction of the three-dimensional agglomerates at the narrow portion.

13. The microfluidic method according to claim 9, wherein the first portion of the first microfluidic channel comprising the at least one narrow portion, is temperature-controlled.

14. The microfluidic method according to claim 9, wherein after step c), the following step is performed:
d) conveying the split individual structures and/or agglomerate fragments through the first retaining element via a second portion of the first microfluidic channel to a second retaining element, whose pores have a diameter smaller than the diameter of the split individual structures and agglomerate fragments such that they are retained, wherein the split individual structures and/or agglomerate fragments are conveyed, via a second rinsing liquid.

15. The microfluidic method according to one claim 9, wherein after step d), the following step is performed:
e) conveying the individual structures and/or agglomerate fragments back via the first microfluidic channel and discharging the individual structures and/or agglomerate fragments via the first fluidic connection, which serves as an outlet.

16. The microfluidic method according to claim 9, wherein, after step d), the following step is performed:
e′) feeding a second medium via the second microfluidic connection, which serves in as an inlet, and guiding the second medium via a third portion of the first microfluidic channel to an underside of the second retaining element and therethrough, such that the retained individual structures and/or agglomerate fragments on the top side of the second retaining element are transferred with the second medium into a second microfluidic channel, which opens up in a third fluidic connection, and discharging the individual structures and/or agglomerate fragments via the third fluidic connection, which serves as an outlet.

17. The microfluidic method according to claim 9, wherein:
the three-dimensional agglomerates are organoids or spheroids,
the split individual structures are organoid cells or spheroid cells, and
the agglomerate fragments are organoid fragments or spheroid fragments.

18. A control unit for controlling the microfluidic method according to claim 9, by electrical actuation of the at least one valve and/or the at least one pump.

19. A microfluidic cartridge, comprising a microfluidic device according to claim 1.

20. The microfluidic method according to claim 9, wherein the first portion of the first microfluidic channel comprising the at least one narrow portion is heated or cooled.
